(19) United States
(12) Patent Application Publication
(10) Pub. No.: US 2008/0107723 A1
(43) Pub. Date: May 8, 2008

(54) Methods of Treating Pulmonary Distress

(76) Inventor: Walter R. Perkins, Pennington, NJ (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/864,070

(22) Filed: Sep. 28, 2007

Related U.S. Application Data
(60) Provisional application No. 60/847,871, filed on Sep. 28, 2006.

(51) Int. Cl. A61K 31/7036; A61K 9/127; A61P 11/00

(52) U.S. Cl. 424/450; 514/40

(57) ABSTRACT

Provided is a method of increasing the forced expiratory volume in one second (FEV1) in a subject comprising administering to the subject in need thereof a therapeutically effective amount of a liposomal formulation. In some embodiments, the liposomal formulation comprises empty liposomes. Also provided is a method of increasing FEV1 in a subject consisting essentially of administering to the subject a therapeutically effective amount of empty liposomes and pharmaceutical carrier. Additionally, provided is a method of treating cystic fibrosis in a subject comprising administering to the subject a therapeutically effective amount of empty liposomes.

### RELATED APPLICATIONS

This application claims the benefit of priority to U.S. provisional application Ser. No. 60/847,871, filed Sep. 28, 2006.

### BACKGROUND OF THE INVENTION

Cystic fibrosis (CF), also called mucoviscidosis, is an autosomal, recessive, hereditary disease of the exocrine glands. It affects the lungs, sweat glands and the digestive system, causing chronic respiratory and digestive problems. It is caused by mutations in the cystic fibrosis transmembrane conductance regulator (CFTR) protein. It is the most common fatal autosomal recessive diseases amongst Caucasians.

The first manifestation of CF is sometimes meconium ileus, occurring in 16% of infants who develop CF. Other symptoms of CF manifest during early childhood. Both lungs and pancreas produce abnormally viscous mucus. This mucus begins to build up and starts to clog the opening to the pancreas and the lungs. Pulmonary problems start from the constant presence of thick, sticky mucus and are one of the most serious complications of CF. The standard for measuring pulmonary function is forced expiratory volume in one second (FEV1). FEV1 is often expressed as a percent of that predicted for a healthy individual based on height, weight, gender and age. Cystic fibrosis patients often have low FEV1 values compared to their healthier counterparts

Daily aerosol breathing treatments are very commonly prescribed for CF patients. Aerosolized medicines commonly given include albuterol, ipratropium bromide and Pulmozyme to loosen secretions and decrease inflammation. Pulmozyme (recombinant human Dnase) administered by inhalation shows an improvement in FEV1 of approximately 10%. Pulmozyme acts by breaking up the fibrous DNAs in the respiratory mucoid system. Tobramycin administered by inhalation has also improved FEV1 by approximately 10%, thought mainly to be due to the substantial reduction in colony forming units (CFU) of about 2 log units.

CF patients are typically hospitalized somewhat regularly, often every 6 months depending on the severity of the case. New methods for increasing pulmonary functions of cystic fibrosis patients which are simple and easy to administer are needed.

### SUMMARY OF THE INVENTION

It is an object to increase the forced expiratory volume in one second (FEV1) in a subject comprising administering to the subject in need thereof a therapeutically effective amount of a liposomal formulation.

It is another object to increase the FEV1 in a subject consisting essentially of administering to the subject in need thereof a therapeutically effective amount of a empty liposomes.

It is another object to treat cystic fibrosis in a subject comprising administering to the subject in need thereof a therapeutically effective amount of a empty liposomes.

It is another object to treat cystic fibrosis in a subject consisting essentially of administering to the subject in need thereof a therapeutically effective amount of a empty liposomes.

The subject invention results in part from the realization that liposomal formulations comprising ineffective concentrations of antiinfectives increased FEV1 in cystic fibrosis patients without reducing CFU leading one to believe that the antiinfective is not necessary to increase FEV1, i.e. that treatment with liposomes alone resulted in increased FEV1.

In one aspect, the disclosure features a method of increasing the forced expiratory volume in one second (FEV1) in a subject comprising administering to the subject in need thereof a therapeutically effective amount of a liposomal formulation.

In a further embodiment, the liposomal formulation does not comprise surfactant agents. In a further embodiment, the liposomal formulation comprises empty liposomes.

In a further embodiment, the liposomal formulation comprises a bioactive agent. In a further embodiment, the bioactive agent is an antiinfective. In a further embodiment, the bioactive agent is an aminoglycoside. In a further embodiment, the bioactive agent is amikacin, tobramycin, or gentamicin. In a further embodiment, the bioactive agent is amikacin. In a further embodiment, the bioactive agent is encapsulated within a liposome. In a further embodiment, the bioactive agent is free.

In a further embodiment, the liposomal formulation comprises empty liposomes and a bioactive agent. In a further embodiment, the bioactive agent is an antiinfective. In a further embodiment, the bioactive agent is an aminoglycoside. In a further embodiment, the bioactive agent is amikacin, tobramycin, or gentamicin. In a further embodiment, the bioactive agent is amikacin. In a further embodiment, the bioactive agent is encapsulated within a liposome. In a further embodiment, the bioactive agent is free.

In a further embodiment, the FEV1 is increased by 5 to 20%, 5 to 15%, or by 10%.

In a further embodiment, the liposomal formulation comprises lipids selected from the group consisting of phospholipids, tocopherols, sterols, glycoproteins, and mixtures thereof. In a further embodiment, the liposomal formulation comprises lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleoylphosphatidylcholine (DOPC), dimyristoylphosphatidylcholine (DMPC), dimyristoylphosphatidylglycerol (DMPG), dipalmitoylphosphatidylglycerol (DPPG), distearoylphosphatidylcholine (DSPQ), distearoylphosphatidylglycerol (DSPG), dioleoylphosphatidyl-ethanolamine (DOPE), palmitoylstearoylphosphatidyl-choline (PSPC), palmitoylstearolphosphatidylglycerol (PSPG), mono-oleoyl-phosphatidylethanolarnine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1, 2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and combinations thereof.

In a further embodiment, the liposomal formulation comprises a phospholipid. In a further embodiment, the liposomal formulation comprises DPPC. In a further embodiment, the liposomal formulation comprises a phospholipid and a sterol. In a further embodiment, the liposomal formulation comprises DPPC and cholesterol. In a further embodiment, the liposomal formulation comprises DPPC:cholesterol in a 1:1 to 3:1 ratio by weight. In a further embodiment, the liposomal formulation comprises DPPC:cholesterol in a 2:1 ratio by weight.

In a further embodiment, the liposomal formulation comprises 500 mg of DPPC and 250 mg of cholesterol. In a further embodiment, the liposomal formulation comprises 250 mg of DPPC and 125 mg of cholesterol.

In a further embodiment, the liposomal formulation is administered twice a day. In a further embodiment, the liposomal formulation is administered once a day.

In a further embodiment, the liposomal formulation is at an aqueous concentration of 10-100 mg/mL, 25-75 mg/mL, 40-50 mg/mL, or 45 mg/mL.

In a further embodiment, 150-700 mg, 200-600 mg, 500 mg, or 250 mg of the liposomal formulation is administered to the subject in a single dose.

In a further embodiment, the liposomal formulation is administered over a treatment period of more than 3, 7, 14, or 21 days.

In another aspect, the disclosure features a method of increasing the forced expiratory volume in one second (FEV1) in a subject consisting essentially of administering to the subject in need thereof a therapeutically effective amount of empty liposomes and a pharmaceutical carrier.

In a further embodiment, the FEV1 is increased by 5 to 20%, 5 to 15%, or 10%.

In a further embodiment, the liposomes comprise lipids selected from the group consisting of phospholipids, tocopherols, sterols, glycoproteins, and mixtures thereof. In a further embodiment, the empty liposomes comprise lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleoylphosphatidylcholine (DOPC), dimyristoylphosphatidylcholine (DMPC), dimyristoylphosphatidylglycerol (DMPG), dipalmitoylphosphatidylglycerol (DPPG), distearoylphosphatidylcholine (DSPQ), distearoylphosphatidylglycerol (DSPG), dioleoylphosphatidyl-ethanolamine (DOPE), palmitoylstearoylphosphatidyl-choline (PSPC), palmitoylstearolphosphatidylglycerol (PSPG), mono-oleoyl-phosphatidylethanolarnine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1, 2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and combinations thereof.

In a further embodiment, the liposomes comprise a phospholipid. In a further embodiment, the liposomes comprise DPPC. In a further embodiment, the liposomes comprise a phospholipid and a sterol. In a further embodiment, the liposomes comprise DPPC and cholesterol. In a further embodiment, the liposomes comprise DPPC:cholesterol in a 1:1 to 3:1 ratio by weight, or in a 2:1 ratio by weight.

In a further embodiment, the liposomes comprise 500 mg of DPPC and 250 mg of cholesterol. In a further embodiment, the liposomes comprise 250 mg of DPPC and 125 mg of cholesterol.

In a further embodiment, the liposomes are administered twice a day or once a day.

In a further embodiment, the liposomes are at an aqueous concentration of 10-100 mg/mL, 25-75 mg/mL, 40-50 mg/mL, or 45 mg/mL.

In a further embodiment, 150-700 mg, 200-600 mg, 500 mg, or 250 mg of liposomes are administered to the subject in a single dose.

In a further embodiment, the liposomes are administered over a treatment period of more than 3, 7, 14, or 21 days.

In another aspect, the disclosure features a method of treating cystic fibrosis in a subject comprising administering to a subject in need thereof a therapeutically effective amount of empty liposomes.

In a further embodiment, the method further comprises administering a bioactive agent. In a further embodiment, the bioactive agent is an antiinfective. In a further embodiment, the bioactive agent is an aminoglycoside. In a further embodiment, the bioactive agent is amikacin, tobramycin, or gentamicin. In a further embodiment, the bioactive agent is amikacin. In a further embodiment, the bioactive agent is encapsulated within a liposome. In a further embodiment, the bioactive agent is free.

In a further embodiment, the liposomes comprise lipids selected from the group consisting of phospholipids, tocopherols, sterols, glycoproteins, and mixtures thereof. In a further embodiment, the liposomes comprise lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleoylphosphatidylcholine (DOPC), dimyristoylphosphatidylcholine (DMPC), dimyristoylphosphatidylglycerol (DMPG), dipalmitoylphosphatidylglycerol (DPPG), distearoylphosphatidylcholine (DSPQ), distearoylphosphatidylglycerol (DSPG), dioleoylphosphatidyl-ethanolamine (DOPE), palmitoylstearoylphosphatidyl-choline (PSPC), palmitoylstearolphosphatidylglycerol (PSPG), mono-oleoyl-phosphatidylethanolarnine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1, 2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and combinations thereof.

In a further embodiment, the liposomes comprise a phospholipid. In a further embodiment, the liposomes comprise DPPC. In a further embodiment, the liposomes comprise a phospholipid and a sterol. In a further embodiment, the liposomes comprise dipalmitoylphosphatidylcholine (DPPC) and cholesterol.

In a further embodiment, the liposomes comprise DPPC:cholesterol in a 1:1 to 3:1 ratio by weight. In a further embodiment, the liposomes comprise DPPC:cholesterol in a 2:1 ratio by weight.

In a further embodiment, the liposomes comprise 500 mg of DPPC and 250 mg of cholesterol. In a further embodiment, the liposomes comprise 250 mg of DPPC and 125 mg of cholesterol.

In a further embodiment, the liposomes are administered twice a day. In a further embodiment, the liposomes are administered once a day.

In a further embodiment, the liposomes are at an aqueous concentration of 10-100 mg/mL, 25-75 mg/mL, 40-50 mg/mL, or 45 mg/mL.

In a further embodiment, 150-700 mg, 200-600 mg, 500 mg, or 250 mg of liposomes are administered to the subject in a single dose.

In a further embodiment, the liposomes are administered over a treatment period of more than 3, 7, 14, or 21 days.

In a further embodiment, the subject is a primate, bovine, ovine, equine, porcine, rodent, feline, or canine. In a further embodiment, the subject is a human.

In another aspect, the disclosure features a method of treating cystic fibrosis in a subject consisting essentially of administering to a subject in need thereof a therapeutically effective amount of empty liposomes and a pharmaceutical carrier.

In a further embodiment, the liposomes comprise lipids selected from the group consisting of phospholipids, tocopherols, sterols, glycoproteins, and mixtures thereof. In a further embodiment, the liposomes comprise lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleoylphosphatidylcholine (DOPC), dimyristoylphosphatidylcholine (DMPC), dimyristoylphosphatidylglycerol (DMPG), dipalmitoylphosphatidylglycerol (DPPG), distearoylphosphatidylcholine (DSPQ), distearoylphosphatidylglycerol (DSPG), dioleoylphosphatidyl-ethanolamine (DOPE), palmitoylstearoylphosphatidyl-choline (PSPC), palmitoylstearolphosphatidylglycerol (PSPG), mono-oleoyl-phosphatidylethanolarnine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1, 2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and combinations thereof.

In a further embodiment, the liposomes comprise a phospholipid. In a further embodiment, the liposomes comprise DPPC. In a further embodiment, the liposomes comprise a phospholipid and a sterol. In a further embodiment, the empty liposomes comprise DPPC and cholesterol.

In a further embodiment, the liposomes comprise DPPC:cholesterol in a 1:1 to 3:1 ratio by weight. In a further embodiment, the liposomes comprise DPPC:cholesterol in a 2:1 ratio by weight. In a further embodiment, the liposomes comprise 500 mg of DPPC and 250 mg of cholesterol. In a further embodiment, the liposomes comprise 250 mg of DPPC and 125 mg of cholesterol.

In a further embodiment, the liposomes are administered twice a day. In a further embodiment, the liposomes are administered once a day.

In a further embodiment, the liposomes are at an aqueous concentration of 10-100 mg/mL, 25-75 mg/mL, 40-50 mg/mL, or 45 mg/mL.

In a further embodiment, 150-700 mg, 200-600 mg, 500 mg, or 250 mg of liposomes are administered to the subject in a single dose.

In a further embodiment, the liposomes are administered over a treatment period of more than 3, 7, 14, or 21 days.

In a further embodiment, the subject is a primate, bovine, ovine, equine, porcine, rodent, feline, or canine. In a further embodiment, the subject is a human.

These embodiments of the present invention, other embodiments, and their features and characteristics, will be apparent from the description, drawings and claims that follow.

### DETAILED DESCRIPTION OF THE INVENTION

### I. Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e. to at least one) of the grammatical object of the article. By way of example, “an element” means one element or more than one element.

The term “bioactive agent” as used herein refers to small molecules or macromolecules with biological activity such as drugs or prodrugs. Since it is believed that FEV1 in a cystic fibrosis patient increases with treatment of lipids, lipids are considered bioactive agents. Therefore, when it is said that the formulations lack an additional bioactive agent, it means it lacks an additional bioactive agent to the lipids. Bioactive agent, as used herein, does not include pharmaceutical excipients.

The term “bioavailable” is art-recognized and refers to a form of the subject invention that allows for it, or a portion of the amount administered, to be absorbed by, incorporated to, or otherwise physiologically available to a subject or patient to whom it is administered.

The terms “comprise” and “comprising” are used in the inclusive, open sense, meaning that additional elements may be included.

The term “consisting” is used to limit the elements to those specified except for impurities ordinarily associated therewith.

The term “consisting essentially of” is used to limit the elements to those specified and those that do not materially affect the basic and novel characteristics of the material or steps.

The term “empty” as used herein to describe liposomes refers to liposomes without encapsulated bioactive agent. An empty liposome may still of course have other things encapsulated along the lines of a pharmaceutical carrier such as, for example, water.

The term “free” as used herein to describe bioactive agents refers to bioactive agents unencapsulated by liposomes.

The term “including” is used herein to mean “including but not limited to”. “Including” and “including but not limited to” are used interchangeably.

The term “mammal” is known in the art, and exemplary mammals include humans, primates, bovines, porcines, canines, felines, and rodents (e.g., mice and rats).

A “patient,” “subject” or “host” to be treated by the subject method may mean either a human or non-human animal.

The term “pharmaceutically-acceptable salts” is art-recognized and refers to the relatively non-toxic, inorganic and organic acid addition salts of compounds, including, for example, those contained in compositions of the present invention.

The term “prodrug” is art-recognized and is intended to encompass compounds which, under physiological conditions, are converted into the antibacterial agents of the present invention. A common method for making a prodrug is to select moieties which are hydrolyzed under physiological conditions to provide the desired compound. In other embodiments, the prodrug is converted by an enzymatic activity of the host animal or the target bacteria.

The term “single dose” is art-recognized and refers to a period of time during which the lipid formulation is being administered, or to the amount of lipid formulation given in that period of time. A single dose may last several seconds, minutes, or hours.

The term “surfactant agent” is art-recognized and refers to agents that facilitate lipid exchange to surfaces. For example, surfactant agents include detergents such as tyloxapol, peptides such as KL₄, and surfactant proteins such as SP-B or SP-C.

The term “treating” is art-recognized and refers to curing as well as ameliorating at least one symptom of any condition or disorder.

### II. Introduction

Lipid formulations have been used for the treatment of pulmonary distress. The lipid formulations comprise additional reagents such as a detergent, peptide, or surfactant protein. For example, Anzueto et al. has reported that Exosurf manufactured by Glaxo Wellcome, Inc. increases the FEV1 in patients with bronchitis. Anzueto, A. et al. JAMA 1997, 278(17), 1426-1431 Exosurf comprises DPPC and the detergent tyloxapol. Other lipid formulations such as Curosurf and Survanta comprise DPPC and surfactant proteins B and C. Discovery Labs is developing an artificial surfactant comprising DPPC and the cationic peptide KL4 which has detergent like properties. In all these cases, DPPC can spread to the air-water interface to lower surface tension and help open airways more easily. Patients with CF are deficient in surfactant and adding some helps with pulmonary function. Also, by spreading to surfaces lipids may coat (lubricate) mucous plugs in CF patients and allow easier expression and clearance of the mucous.

It is generally accepted that without one of these additional reagents (e.g. detergent, peptide, or surfactant protein) spreading will not occur.

It has now surprisingly been found that the liposomal formulations of the present invention which lack additional reagents effectively increase FEV1 in cystic fibrosis patients. The liposomes of the present invention are regarded as “tough” liposomes and would not be expected to improve lung function as they have. Not wanting to be bound by theory, one possible explanation for this phenomena is that the liposomes are binding/adhering to the mucous surfaces and allowing easier expression and clearance. The liposomes may also be coating airway surfaces and providing lubrication.

### III. Lipids

The lipids used in the lipid formulations can be synthetic, semi-synthetic or naturally-occurring lipids, including phospholipids, tocopherols, sterols, fatty acids, glycoproteins such as albumin, negatively-charged lipids and cationic lipids. In terms of phospholipids, they could include such lipids as phosphatidylcholine (PC), phosphatidylglycerol (PG), phosphatidylinositol (PI), phosphatidylserine (PS), phosphatidylethanolamine (PE), and phosphatidic acid (PA). The egg counterparts, egg phosphatidylcholine (EPC), egg phosphatidylglycerol (EPG), egg phosphatidylinositol (EPI), egg phosphatidylserine (EPS), egg phosphatidylethanolamine (EPE), and egg phosphatidic acid (EPA); the soya counterparts, soy phosphatidylcholine (SPC), SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the I position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include dipalmitoylphophatidylcholine (DPPC), a major constituent of naturally-occurring lung surfactant. Other examples include dioleoylphosphatidylcholine (DOPC), dimyristoylphosphatidylcholine (DMPC), dimyristoylphosphatidylglycerol (DMPG), dipalmitoylphosphatidylglycerol (DPPG), distearoylphosphatidylcholine (DSPQ), distearoylphosphatidylglycerol (DSPG), dioleoylphosphatidyl-ethanolamine (DOPE) and mixed phospho lipids like palmitoylstearoylphosphatidyl-choline (PSPC) and palmitoylstearolphosphatidylglycerol (PSPG), and single acylated phospholipids like mono-oleoyl-phosphatidylethanolarnine (MOPE).

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

The cationic lipids used can include ammonium salts of fatty acids, phospholipids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1, 2-bis(oleoyloxy)-3-(trimethylammonio)propane (DOTAP).

The negatively-charged lipids which can be used include phosphatidyl-glycerols (PGs), phosphatidic acids (PAs), phosphatidylinositols (Pls) and the phosphatidyl serines (PSs). Examples include DMPG, DPPG, DSPG, DMPA, DPPA, DSPA, DMPI, DPPI, DSPI, DMPS, DPPS and DSPS.

### IV. Liposomes

In one embodiment of the present invention, the lipid formulations comprise liposomes. Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer). The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes can be produced by a variety of methods (for a review, see, e.g., Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation.

Unilamellar vesicles can be produced from MLVs by a number of techniques, for example, the extrusion of Cullis et al. (U.S. Pat. No. 5,008,050) and Loughrey et al. (U.S. Pat. No. 5,059,421)). Sonication and homogenization cab be so used to produce smaller unilamellar liposomes from larger liposomes (see, for example, Paphadjopoulos et al. (1968); Deamer and Uster (1983); and Chapman et al. (1968)).

The original liposome preparation of Bangham et al. (J. Mol. Biol., 1965, 13:238-252) involves suspending phospholipids in an organic solvent which is then evaporated to dryness leaving a phospholipid film on the reaction vessel. Next, an appropriate amount of aqueous phase is added, the 60 mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys, Acta., 1967, 135:624-638), and large unilamellar vesicles.

Techniques for producing large unilamellar vesicles (LUVs), such as, reverse phase evaporation, infusion procedures, and detergent dilution, can be used to produce liposomes. A review of these and other methods for producing liposomes may be found in the text Liposomes, Marc Ostro, ed., Marcel Dekker, Inc., New York, 1983, Chapter 1, the pertinent portions of which are incorporated herein by reference. See also Szoka, Jr. et al., (1980, Ann. Rev. Biophys. Bioeng., 9:467), the pertinent portions of which are also incorporated herein by reference.

Other techniques that are used to prepare vesicles include those that form reverse-phase evaporation vesicles (REV), Papahadjopoulos et al., U.S. Pat. No. 4,235,871. Another class of liposomes that may be used are those characterized as having substantially equal lamellar solute distribution. This class of liposomes is denominated as stable plurilamellar vesicles (SPLV) as defined in U.S. Pat. No. 4,522,803 to Lenk, et al. and includes monophasic vesicles as described in U.S. Pat. No. 4,588,578 to Fountain, et al. and frozen and thawed multilamellar vesicles (FATMLV) as described above.

A variety of sterols and their water soluble derivatives such as cholesterol hemisuccinate have been used to form liposomes; see specifically Janoff et al., U.S. Pat. No. 4,721,612, issued Jan. 26, 1988, entitled “Steroidal Liposomes.” Mayhew et al., PCT Publication No. WO 85/00968, published Mar. 14, 1985, described a method for reducing the toxicity of drugs by encapsulating them in liposomes comprising alpha-tocopherol and certain derivatives thereof. Also, a variety of tocopherols and their water soluble derivatives have been used to form liposomes, see Janoff et al., PCT Publication No. 87/02219, published Apr. 23, 1987, entitled “Alpha Tocopherol-Based Vesicles”.

The liposomes are comprised of particles with a mean diameter of approximately 0.01 microns to approximately 3.0 microns, preferably in the range about 0.1 to 1.0 microns, and even more preferably in the range of about 0.1 to 0.5 microns.

### V. Antiinfectives

Antiinfectives are agents that act against infections, such as bacterial, mycobacterial, fungal, viral or protozoal infections. Antiinfectives covered by the invention include but are not limited to aminoglycosides (e.g., streptomycin, gentamicin, tobramycin, amikacin, netilmicin, kanamycin, and the like), tetracyclines (such as chlortetracycline, oxytetracycline, methacycline, doxycycline, minocycline and the like), sulfonamides (e.g., sulfanilamide, sulfadiazine, sulfamethaoxazole, sulfisoxazole, sulfacetamide, and the like), paraaminobenzoic acid, diaminopyrimidines (such as trimethoprim, often used in conjunction with sulfamethoxazole, pyrazinamide, and the like), quinolones (such as nalidixic acid, cinoxacin, ciprofloxacin and norfloxacin and the like), penicillins (such as penicillin G, penicillin V, ampicillin, amoxicillin, bacampicillin, carbenicillin, carbenicillin indanyl, ticarcillin, azlocillin, mezlocillin, piperacillin, and the like), penicillinase resistant penicillin (such as methicillin, oxacillin, cloxacillin, dicloxacillin, nafcillin and the like), first generation cephalosporins (such as cefadroxil, cephalexin, cephradine, cephalothin, cephapirin, cefazolin, and the like), second generation cephalosporins (such as cefaclor, cefamandole, cefonicid, cefoxitin, cefotetan, cefuroxime, cefuroxime axetil; cefinetazole, cefprozil, loracarbef, ceforanide, and the like), third generation cephalosporins (such as cefepime, cefoperazone, cefotaxime, ceftizoxime, ceftriaxone, ceftazidime, cefixime, cefpodoxime, ceftibuten, and the like), other beta-lactams (such as imipenem, meropenem, aztreonam, clavulanic acid, sulbactam, tazobactam, and the like), betalactamase inhibitors (such as clavulanic acid), chlorampheriicol, macrolides (such as erythromycin, azithromycin, clarithromycin, and the like), lincomycin, clindamycin, spectinomycin, polymyxin B, polymixins (such as polymyxin A, B, C, D, E1 (colistin A), or E2, colistin B or C, and the like) colistin, vancomycin, bacitracin, isoniazid, rifampin, ethambutol, ethionamide, aminosalicylic acid, cycloserine, capreomycin, sulfones (such as dapsone, sulfoxone sodium, and the like), clofazimine, thalidomide, or any other antibacterial agent that can be lipid encapsulated. Antiinfectives can include antifungal agents, including polyene antifungals (such as amphotericin B, nystatin, natamycin, and the like), flucytosine, imidazoles (such as n-ticonazole, clotrimazole, econazole, ketoconazole, and the like), triazoles (such as itraconazole, fluconazole, and the like), griseofulvin, terconazole, butoconazole ciclopirax, ciclopirox olamine, haloprogin, tolnaftate, naftifine, terbinafine, or any other antifungal that can be lipid encapsulated or complexed. Discussion and the examples are directed primarily toward amikacin but the scope of the application is not intended to be limited to this antiinfective. Combinations of drugs can be used.

Particularly preferred antiinfectives include the aminoglycosides, the quinolones, the polyene antifungals and the polymyxins.

Also included as suitable antiinfectives used in the liposomal formulations are pharmaceutically acceptable addition salts and complexes of antiinfectives. In cases wherein the compounds may have one or more chiral centers, unless specified, the present invention comprises each unique racemic compound, as well as each unique nonracemic compound.

In cases in which the antiinfectives have unsaturated carbon-carbon double bonds, both the cis (Z) and trans (E) isomers are within the scope of this invention. In cases wherein the antiinfectives may exist in tautomeric forms, such as keto-enol tautomers, such as 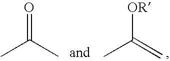
each tautomeric form is contemplated as being included within this invention, whether existing in equilibrium or locked in one form by appropriate substitution with R′. The meaning of any substituent at any one occurrence is independent of its meaning, or any other substituent's meaning, at any other occurrence.

Also included as suitable antiinfectives used in the liposomal formulations are prodrugs of the platinum compounds. Prodrugs are considered to be any covalently bonded carriers which release the active parent compound in vivo.

### VI. Dosages

The dosage of any formulation of the present invention will vary depending on the symptoms, age and body weight of the patient, the nature and severity of the disorder to be treated or prevented, the route of administration, and the form of the subject composition. Any of the subject compositions may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the composition, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

Generally, the lipid formulation is administered to the subject in need thereof on a daily basis. Daily, the subject may receive at least a single dose of lipid formulation which may last several seconds, minutes, or hours. In one embodiment, the single dose administers 300-700 mg of lipid. In a further embodiment, the single dose administers 500 mg of lipid.

### VII. Pharmaceutical Formulation

The pharmaceutical formulation of the liposomal formulation of the present invention may be comprised of either an aqueous dispersion of the liposomal formulations, or a dehydrated powder containing the liposomal formulations. The formulation may contain lipid excipients to form the liposomes, and salts/buffers to provide the appropriate osmolarity and pH. The dry powder formulations may contain additional excipients to prevent the leakage of encapsulated components during the drying and potential milling steps needed to create a suitable particle size for inhalation (i.e., about 1-5 μm). Such excipients are designed to increase the glass transition temperature of the liposomal formulation. The pharmaceutical excipient may be a liquid or solid filler, diluent, solvent or encapsulating material, involved in carrying or transporting any subject composition or component thereof from one organ, or portion of the body, to another organ, or portion of the body. Each excipient must be “acceptable” in the sense of being compatible with the subject composition and its components and not injurious to the patient. Suitable excipients include trehalose, raffinose, mannitol, sucrose, leucine, trileucine, and calcium chloride. Examples of other suitable excipients include (1) sugars, such as lactose, and glucose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The lipid formulation is generally an aqueous solution of lipids. In one embodiment, the concentration of lipid in the lipid formulation is anywhere from 50-100 mg/mL. In a further embodiment, the lipid concentration is anywhere from 75-80 mg/mL.

### VIII. Inhalation Device

The liposomal formulations of the present invention may be used in any dosage dispensing device adapted for intranasal administration. The device should be constructed with a view to ascertaining optimum metering accuracy and compatibility of its constructive elements, such as container, valve and actuator with the nasal formulation and could be based on a mechanical pump system, e.g., that of a metered-dose nebulizer, dry powder inhaler, soft mist inhaler, or a nebulizer. Due to the large administered dose, preferred devices include jet nebulizers (e.g., PARI LC Star, AKITA), soft mist inhalers (e.g., PARI e-Flow), and capsule-based dry powder inhalers (e.g., PH&T Turbospin). Suitable propellants may be selected among such gases as fluorocarbons, hydrocarbons, nitrogen and dinitrogen oxide or mixtures thereof.

The inhalation delivery device can be a nebulizer or a metered dose inhaler (MDI), or any other suitable inhalation delivery device known to one of ordinary skill in the art. The device can contain and be used to deliver a single dose of the liposomal formulations or the device can contain and be used to deliver multi-doses of the compositions of the present invention.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device may comprise a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer may be employed to ensure the sizing of particles is optimal for positioning of the particle within, for example, the pulmonary membrane.

A metered dose inhalator (MDI) may be employed as the inhalation delivery device for the compositions of the present invention. This device is pressurized (pMDI) and its basic structure comprises a metering valve, an actuator and a container. A propellant is used to discharge the formulation from the device. The composition may consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. Traditional chloroflourocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To insure accuracy of dosing, the delivery of the formulation may be programmed via a microprocessor to occur at a certain point in the inhalation cycle. The MDI may be portable and hand held.

### EXEMPLIFICATION

### Example 1

A study was conducted where several patients with cystic fibrosis were administered lipid formulations comprising 500 mg of DPPC, 250 mg of cholesterol, and 500 mg of entrapped amikacin. The lipid formulation was administered daily for 14 days. The effects of the lipid formulation on FEV1 and colony forming units (CFU) appear in Tables 1 and 2, respectively.

TABLE 1
The effect of the lipid formulation on FEV1.

| | | | FEV1 | | | FEV1 |
|---|---|---|---|---|---|---|
| | FEV1 | | increase | | | increase |
| | before | FEV1 after | after | FEV1 | FEV1 after | after |
| | treatment | treatment | treatment | before | treatment | treatment |
| Patient | (L) | (L) | (mL) | treatment % | % | % |
| A | 1.64 | 1.81 | 170 | 59 | 65 | 6 |
| B | 5.09 | 5.49 | 400 | 114.6 | 123.6 | 9 |
| C | 2.4 | 2.67 | 270 | 76.6 | 85.1 | 8.5 |
| D | 0.88 | 1.22 | 340 | 31 | 43 | 12 |
| | | | | | Ave. | 8.9 |

TABLE 2
The effect of the lipid formulation on CFU.

| | | | log(CFU) | log(CFU) | |
|---|---|---|---|---|---|
| | CFU before | CFU after | before | after | Change in |
| Patient | treatment | treatment | treatment | treatment | log(CFU) |
| A | 2.00E+7 | 4.80E+6 | 7.30 | 6.68 | −0.62 |
| B | 1.00E+5 | 1.00E+6 | 5.00 | 6.00 | +1.00 |
| C | 1.00E+5 | 2.00E+7 | 5.00 | 7.30 | +2.30 |
| D | 2.00E+8 | 2.00E+8 | 8.3 | 8.30 | 0 |
| | | | | Ave. | +0.67 |

The study indicates that the treatment resulted in about a 10% improvement in FEV1 without any meaningful reduction in CFU. It has been reported that treatment with free aminoglycoside by inhalation of CF patients caused a substantial reduction in CFU (approximately 2 log units) as well as approximately a 10% improvement in FEV1. The improved FEV1 values were explained as an overall pulmonary function improvement resulting from the antiinfective activity of the aminoglycoside.

The present study shows that FEV1 improvement was achieved even though there was not enough aminoglycoside to cause noticeable antiinfective activity. This strongly indicates that the lipids are the main cause for FEV1 improvement. Not wanting to be bound by theory, one explanation of this unexpected phenomena is that the liposome associates with the DNA causing condensation of DNA. Another explanation may be that the liposome lubricates the airways.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of increasing the forced expiratory volume in one second (FEV1) in a subject comprising administering to the subject in need thereof a therapeutically effective amount of a liposomal formulation.

2. The method of claim 1, wherein the liposomal formulation does not comprise surfactant agents.

3. The method of claim 1, wherein the liposomal formulation comprises empty liposomes.

4. The method of claim 1, wherein the liposomal formulation comprises a bioactive agent.

5. The method of claim 4, wherein the bioactive agent is an antiinfective.

6. The method of claim 4, wherein the bioactive agent is an aminoglycoside.

7. The method of claim 4, wherein the bioactive agent is amikacin, tobramycin, or gentamicin.

8. The method of claim 4, wherein the bioactive agent is amikacin.

9. The method of claim 4 wherein the bioactive agent is encapsulated within a liposome.

10. The method of claim 4 wherein the bioactive agent is free.

11. The method of claim 1, wherein the liposomal formulation comprises empty liposomes and a bioactive agent.

12-17. (canceled)

18. The method of claim 1, wherein the FEV1 is increased by 5 to 20%.

19-20. (canceled)

21. The method of claim 1, wherein the liposomal formulation comprises lipids selected from the group consisting of phospholipids, tocopherols, sterols, glycoproteins, and mixtures thereof.

22. The method of claim 1, wherein the liposomal formulation comprises lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleoylphosphatidylcholine (DOPC), dimyristoylphosphatidylcholine (DMPC), dimyristoylphosphatidylglycerol (DMPG), dipalmitoylphosphatidylglycerol (DPPG), distearoylphosphatidylcholine (DSPQ), distearoylphosphatidylglycerol (DSPG), dioleoylphosphatidyl-ethanolamine (DOPE), palmitoylstearoylphosphatidyl-choline (PSPC), palmitoylstearolphosphatidylglycerol (PSPG), mono-oleoyl-phosphatidylethanolarnine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1, 2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and combinations thereof.

23. The method of claim 1, wherein the liposomal formulation comprises a phospholipid.

24. The method of claim 1, wherein the liposomal formulation comprises DPPC.

25. The method of claim 1, wherein the liposomal formulation comprises a phospholipid and a sterol.

26. The method of claim 1, wherein the liposomal formulation comprises DPPC and cholesterol.

27. The method of claim 1, wherein the liposomal formulation comprises DPPC cholesterol in a 1:1 to 3:1 ratio by weight.

28-30. (canceled)

31. The method of claim 1, wherein the liposomal formulation is administered twice a day.

32. The method of claim 1, wherein the liposomal formulation is administered once a day.

33. The method of claim 1, wherein the liposomal formulation is at an aqueous concentration of 50-100 mg/mL.

34-37. (canceled)

38. The method of claim 1, wherein 150-700 mg of the liposomal formulation is administered to the subject in a single dose.

39-41. (canceled)

42. The method of claim 1, wherein the liposomal formulation is administered over a treatment period of more than 3 days.

43-73. (canceled)

74. A method of treating cystic fibrosis in a subject comprising administering to a subject in need thereof a therapeutically effective amount of empty liposomes.

75. The method of claim 74, further comprising administering a bioactive agent.

76. The method of claim 74, wherein the bioactive agent is an antiinfective.

77. The method of claim 74, wherein the bioactive agent is an aminoglycoside.

78. The method of claim 74, wherein the bioactive agent is amikacin, tobramycin, or gentamicin.

79. The method of claim 74, wherein the bioactive agent is amikacin.

80. The method of claim 75 wherein the bioactive agent is encapsulated within a liposome.

81. The method of claim 75 wherein the bioactive agent is free.

82. The method of claim 74, wherein the liposomes comprise lipids selected from the group consisting of phospholipids, tocopherols, sterols, glycoproteins, and mixtures thereof.

83. The method of claim 74, wherein the liposomes comprises lipids selected from the group consisting of phosphatidylcholine (PC), phosphatidylglycerol (PG), phosphatidylinositol (PI), phosphatidylserine (PS), phosphatidylethanolamine (PE), phosphatidic acid (PA), egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), HEPC, HSPC, dipalmitoylphophatidylcholine (DPPC), dioleoylphosphatidylcholine (DOPC), dimyristoylphosphatidylcholine (DMPC), dimyristoylphosphatidylglycerol (DMPG), dipalmitoylphosphatidylglycerol (DPPG), distearoylphosphatidylcholine (DSPQ), distearoylphosphatidylglycerol (DSPG), dioleoylphosphatidyl-ethanolamine (DOPE), palmitoylstearoylphosphatidyl-choline (PSPC), palmitoylstearolphosphatidylglycerol (PSPG), mono-oleoyl-phosphatidylethanolarnine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, tocopherols, tocopherol hemi-succinates, tocopherol hydrogen sulfates, tocopherol sulfates, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA) and 1, 2-bis(oleoyloxy)-3-(trimethylammonio)propane (DOTAP), DMPG, DPPG, DSPG, DMPA, DPPA, DSPA, DMPI, DPPI, DSPI, DMPS, DPPS, DSPS, an N-acylated phosphorylethanolamine (NAPE), and combinations thereof.

84. The method of claim 74, wherein the liposomes comprise a phospholipid.

85. The method of claim 74, wherein the liposomes comprise DPPC.

86. The method of claim 74, wherein the liposomes comprise a phospholipid and a sterol.

87. The method of claim 74, wherein the liposomes comprise dipalmitoylphosphatidylcholine (DPPC) and cholesterol.

88. The method of claim 74, wherein the liposomes comprise DPPC:cholesterol in a 1:1 to 3:1 ratio by weight.

89-91. (canceled)

92. The method of claim 74, wherein the liposomes are administered twice a day.

93. The method of claim 74, wherein the liposomes are administered once a day.

94. The method of claim 74, wherein the liposomes are at an aqueous concentration of 10-100 mg/mL.

95-97. (canceled)

98. The method of claim 74, wherein 150-700 mg of liposomes are administered to the subject in a single dose.

99-101. (canceled)

102. The method of claim 74, wherein the liposomes are administered over a treatment period of more than 3 days.

103-106. (canceled)

107. The method of claim 74, wherein the subject is a human.

108-135. (canceled)
